(19) United States
(12) Patent Application Publication
(10) Pub. No.: US 2022/0071557 A1
(43) Pub. Date: Mar. 10, 2022

(54) METHODS AND SYSTEMS FOR ACCURATE NOCTURNAL MOVEMENT CLASSIFICATION

(71) Applicant: KONINKLIJKE PHILIPS N.V., ENDHOVEN (NL)

(72) Inventors: Salvatore SAPORITO, Rotterdam (NL); Warner Rudolph Theophile TEN KATE, Waalre (NL); Felipe Maia MASCULO, Eindhoven (NL)

(21) Appl. No.: 17/467,891

(22) Filed: Sep. 7, 2021

Related U.S. Application Data
(60) Provisional application No. 63/075,646, filed on Sep. 8, 2020.

(51) Int. Cl. A61B 5/00; G06F 1/16; H04L 29/08

(52) U.S. Cl. CPC A61B 5/4815; H04L 67/12; G06F 1/163; A61B 5/4809

(57) ABSTRACT

A method for classifying movement of a user during a period of low activity, such as sleep, is provided. The method includes providing a wearable device having a motion sensor; obtaining sensor data from the motion sensor in a buffer having raw sensor data points within a time period; determining whether at least part of the raw sensor data points in the first buffer meets a predefined condition based on a stationarity of motion in the raw sensor data; deriving a low-temporal resolution representation of the raw sensor data points in the first buffer when the at least part of the plurality of raw sensor data points in the first buffer meets the predefined condition based on stationarity; adapting the low-temporal resolution representation based on an estimated effect of a phenomenon on the low-temporal resolution representation; and classifying movement of the user based on the adapted low-temporal resolution representation.

### CROSS-REFERENCE TO PRIOR APPLICATIONS

This application claims the benefit of U.S. Provisional Application No. 63/075,646, filed on 8 Sep. 2020. This application is hereby incorporated by reference herein.

### FIELD OF THE DISCLOSURE

The present disclosure is directed generally to methods and systems for improved movement classification during a period of low activity such as sleep using summarized motion sensor data. More specifically, the present disclosure is directed generally to methods and systems for improving classification accuracy by reducing artifacts related to sporadic, weak motion episodes and bed turns and distinguishing between sporadic and continuous movement episodes using summarized motion sensor data at multiple resolutions.

### BACKGROUND

User behavior and sensor offsets can make it challenging to accurately classify small bodily movements. During the night, people who are sleeping in a bed will turn, changing their body position in bed between lateral, supine and prone positions. Such movements can be detected using body-worn accelerometers. In general, bed turns and other sporadic movements provide information about sleep stages of a subject. Detectable changes in quantity and quality of bed turns (e.g., nocturnal hypokinesia) can be symptomatic of possible health conditions (e.g., Parkinson's disease). Thus, the accurate characterization of nocturnal hypokinesia and other changes in bodily movement during sleep may yield additional clinical value.

Motion sensor data (e.g., accelerometer data) have been widely used for tracking sleep and nocturnal mobility. Sleep tracking is an essential feature of most commercial wearable activity tracker devices. However, the time resolution of the raw signals recorded from the motion sensor (e.g., 50 Hz) implies significant requirements in terms of data storage, data transmission bandwidth, and computational power. Typically, wearable sleep trackers run an algorithm that includes the raw sensor data being sampled, or summarized, into some lower temporal rate, in order to save transmission bandwidth, storage capacity on device, and computational power. This sampled or summarized data can be used to determine whether a subject was sleeping or not. If the raw sensor data has moderate or large signal strengths, the activity classification can be performed accurately. However, when signal strengths are low, the summarized data can hamper adequate classification.

Generally, fixed time resolution approaches for low temporal rate summary signals do not allow the characterization of nocturnal movement episodes, including distinguishing between sporadic and continuous movement episodes (e.g., bed movement versus short awakening and walking).

### SUMMARY OF THE DISCLOSURE

The present disclosure is directed to inventive methods and systems for improved movement classification during periods of low activity such as sleep. Generally, embodiments of the present disclosure are directed to improved methods and systems for distinguishing between sporadic and continuous movement episodes using summarized motion sensor data at multiple resolutions. Applicant has recognized and appreciated that it would be beneficial to accurately discriminate between sporadic and continuous activity for any user-facing device either for monitoring purposes (accurate count awakening episodes in ordinary sleep tracking) or while delivering an intervention (e.g., PowerSleep devices). Applicant has also recognized and appreciated that it would be beneficial to address the overestimated activity levels observed in a low rate summary signal caused by weak motions, together with miscalibration. Various embodiments and implementations herein are directed to deriving and adapting a low-temporal resolution representation of raw sensor data on a wearable device and storing and/or transmitting only this low-temporal resolution representation for further processing for accurate movement classification. The low-temporal resolution representation may include a motion level (e.g., a variance of accelerometer samples in a sliding window).

Generally, in one aspect, a method for classifying movement of a user during a period of low activity is provided. The method includes the steps of providing a wearable device configured to be worn by the user, the wearable device having at least one motion sensor and one or more processors; obtaining, via the one or more processors, raw sensor data from the at least one motion sensor in a first buffer, the first buffer comprising a first plurality of raw sensor data points within a first time period; determining, via the one or more processors, whether at least part of the plurality of raw sensor data points in the first buffer meets a predefined condition based on a stationarity of motion in the raw sensor data; deriving, via the one or more processors, a low-temporal resolution representation of the raw sensor data points in the first buffer when the at least part of the plurality of raw sensor data points in the first buffer meets the predefined condition based on stationarity; adapting, via the one or more processors, the low-temporal resolution representation based on an estimated effect of a phenomenon on the low-temporal resolution representation; and classifying movement of the user based on the adapted low-temporal resolution representation.

According to an embodiment, the method further includes the steps of retrieving historical low-temporal resolution representation data, and estimating the effect of the phenomenon on the low-temporal resolution representation based on the historical low-temporal resolution representation data.

According to an embodiment, the step of determining whether the plurality of raw sensor data points meet the predefined condition includes evaluating the predefined condition using historical low-temporal resolution representation data from the wearable device.

According to an embodiment, the method further includes the steps of storing the low-temporal resolution representation on the one or more processors of the wearable device, and transmitting the low-temporal resolution representation for processing to classify the movement of the user.

According to an embodiment, the method further includes the step of deriving, via the one or more processors, an alternative representation of the raw sensor data points after the step of deriving the low-temporal resolution representation. In embodiments, the method further includes the step of storing the alternative representation on the one or more processors of the wearable device.

According to an embodiment, the method further includes the step of flagging at least part of the first buffer as different when the predefined condition indicates the buffer comprises non-stationary movement.

According to an embodiment, the method further includes the steps of obtaining, via the one or more processors, raw sensor data from the at least one motion sensor in a second buffer, the second buffer comprising a second plurality of raw sensor data points within a second time period, and storing the low-temporal resolution representation in the second buffer. In embodiments, the method further includes the steps of determining a variance of the second plurality of raw sensor data points based on the low-temporal resolution representation stored in the second buffer, and storing the derived and adapted low-temporal resolution representation when the determined variance is below a predetermined threshold value.

According to an embodiment, the method further includes the steps of obtaining, via the one or more processors, raw sensor data from the at least one motion sensor in a second buffer, the second buffer comprising a second plurality of raw sensor data points within a second time period, and storing the low-temporal resolution representation in the second buffer. In embodiments, the method further includes the steps of determining a variance of the second plurality of raw sensor data points based on the low-temporal resolution representation stored in the second buffer, and storing the first plurality of raw sensor data points from the first buffer when the determined variance is above a predetermined threshold value.

Generally, in another aspect, a system for classifying movement of a user during a period of low activity is provided. The system includes a wearable device configured to be worn by the user, the wearable device comprising at least one motion sensor, and one or more processors communicably coupled with the at least one motion sensor. The one or more processors are configured to: obtain raw sensor data from the at least one motion sensor in a first buffer, the first buffer comprising a first plurality of raw sensor data points within a first time period; determine whether at least part of the plurality of raw sensor data points in the first buffer meets a predefined condition based on a stationarity of motion in the raw sensor data; derive a low-temporal resolution representation of the raw sensor data points in the first buffer when the at least part of the plurality of raw sensor data points in the first buffer meets the predefined condition based on stationarity; adapt the low-temporal resolution representation based on an estimated effect of a phenomenon on the low-temporal resolution representation; and classify movement of the user based on the adapted low-temporal resolution representation.

According to an embodiment, the one or more processors are configured to store the low-temporal resolution representation, and transmit the low-temporal resolution representation for processing to classify the movement of the user.

According to an embodiment, the one or more processors are configured to derive an alternative representation of the raw sensor data points based on the low-temporal resolution representation, and store the alternative representation.

According to an embodiment, the one or more processors are configured to obtain raw sensor data from the at least one motion sensor in a second buffer, the second buffer comprising a second plurality of raw sensor data points within a second time period, and store the low-temporal resolution representation in the second buffer. In embodiments, the one or more processors are configured to: determine a variance of the second plurality of raw sensor data points based on the low-temporal resolution representation stored in the second buffer, and store the derived and adapted low-temporal resolution representation when the determined variance is below a predetermined threshold value, or store the first plurality of raw sensor data points from the first buffer when the determined variance is above the predetermined threshold value.

In various implementations, the one or more processors described herein may take any suitable form, such as, one or more processors or microcontrollers, circuitry, one or more controllers, a field programmable gate array (FGPA), or an application-specific integrated circuit (ASIC) configured to execute software instructions. Memory associated with the processor may take any suitable form or forms, including a volatile memory, such as random-access memory (RAM), static random-access memory (SRAM), or dynamic random-access memory (DRAM), or non-volatile memory such as read only memory (ROM), flash memory, a hard disk drive (HDD), a solid-state drive (SSD), or other non-transitory machine-readable storage media. The term “non-transitory” means excluding transitory signals but does not further limit the forms of possible storage. In some implementations, the storage media may be encoded with one or more programs that, when executed on one or more processors and/or controllers, perform at least some of the functions discussed herein. It will be apparent that, in embodiments where the processor implements one or more of the functions described herein in hardware, the software described as corresponding to such functionality in other embodiments may be omitted. Various storage media may be fixed within a processor or may be transportable, such that the one or more programs stored thereon can be loaded into the processor so as to implement various aspects as discussed herein. Data and software, such as the algorithms or software necessary to analyze the data collected by the tags and sensors, an operating system, firmware, or other application, may be installed in the memory.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1A is a schematic illustration of a person wearing a motion sensor for detecting bodily movement during sleep and a system for classifying movement in accordance with aspects of the present disclosure;

FIG. 1B is a schematic illustration of a person wearing a motion sensor for detecting bodily movement during sleep in accordance with aspects of the present disclosure;

FIG. 2 is a schematic graphical depiction of original motion sensor data generated from a motion sensor in accordance with aspects of the present disclosure;

FIG. 3 is a schematic graphical depiction of low-temporal resolution features of the motion sensor data of FIG. 2 in accordance with aspects of the present disclosure;

FIG. 4 is a schematic graphical depiction of rotation induced apparent activities in low-temporal resolution settings of the motion sensor data of FIG. 2 in accordance with aspects of the present disclosure;

FIG. 5 is an example graphical depiction of a full tri-axial accelerometer signal representation recorded during one bed turn in area 5 shown in FIG. 7 in accordance with aspects of the present disclosure;

FIG. 6 is an example graphical depiction of a full tri-axial accelerometer signal representation recorded during sporadic nocturnal activity in area 6 shown in FIG. 7 in accordance with aspects of the present disclosure;

FIG. 7 is an example graphical depiction of low-temporal resolution features, orientation data, and raw accelerometer signals for bodily movement during sleep in accordance with aspects of the present disclosure;

FIG. 8 is an example graphical depiction of low-temporal resolution features, orientation data, and raw accelerometer signals for bodily movement during sleep in accordance with aspects of the present disclosure; and

FIG. 9 is an example process of classifying movement of a user during a period of low activity in accordance with aspects of the present disclosure.

### DETAILED DESCRIPTION OF EMBODIMENTS

The present disclosure describes various embodiments of methods and systems for improving movement classification during a period of low activity such as sleep. More specifically, Applicant has recognized and appreciated that it would be beneficial to reduce sensitivity to potential sensor miscalibration and artifacts related to sporadic, weak motion episodes and bed turns. Additionally, Applicant has recognized and appreciated that it would be beneficial to use multiple time resolution approaches for low temporal rate summary signals to differentiate between sporadic and continuous movement episodes. Advantageously, the multi-scale signal storage provides a technological improvement as the summarized signals are stored and employed until additional details are needed to distinguish between sporadic a continuous movement episodes. Exemplary goals of utilization of certain embodiments of the present disclosure are to use multiple time resolution approaches for low temporal resolution summary signals to reduce the number of sporadic night movements reports to a user/caregiver due to bed turns.

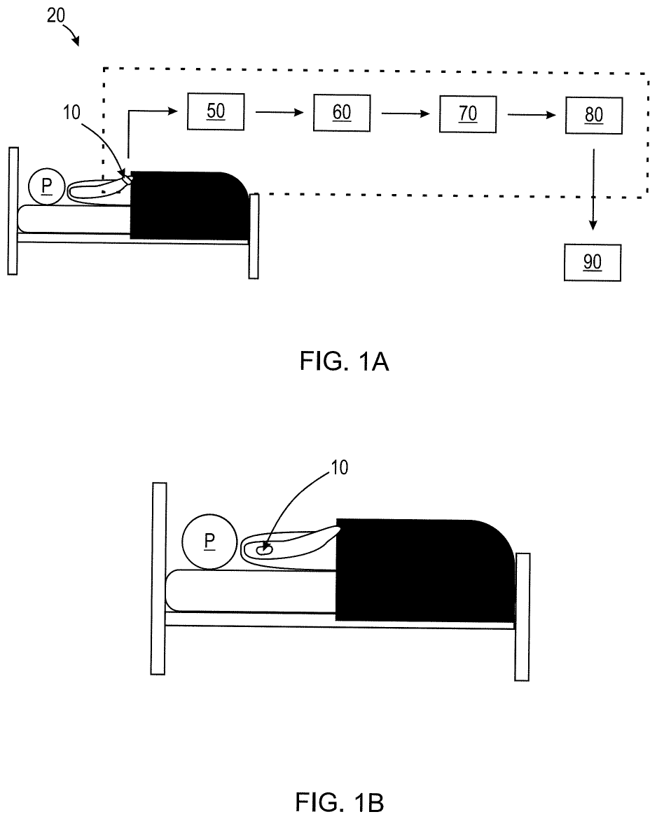

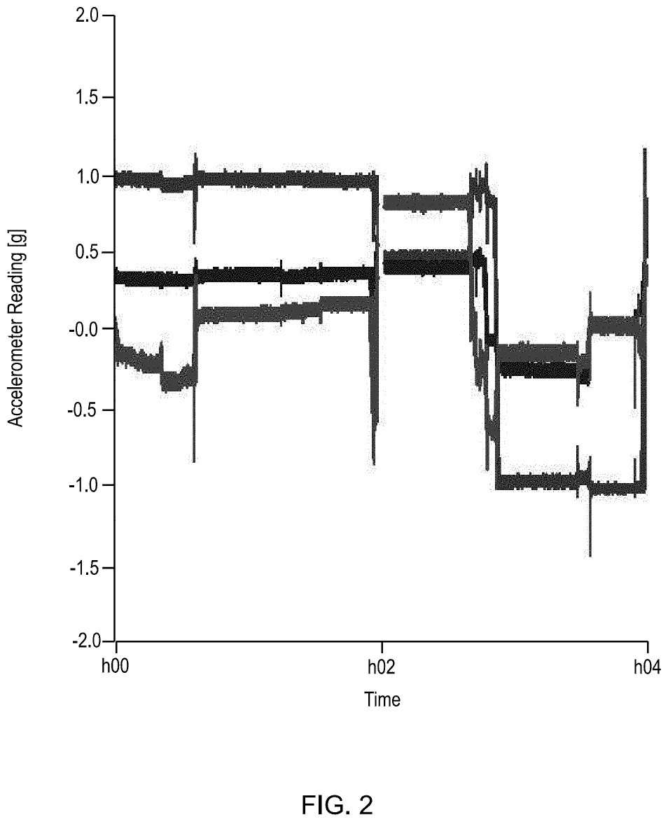

Referring to FIGS. 1A and 1B, schematic depictions are provided of a person P wearing a device 10 comprising one or more motion sensors. The one or more sensors of the device 10 are configured to generate motion data samples indicative of movement of the device 10 when person P is moving while sleeping. FIG. 2 shows raw motion data generated from the one or more sensors of the device in an example embodiment. Although the figures describe device 10 as comprising one or more accelerometers (e.g., a tri-axial accelerometer that measures motion along the x, y, and z axes), it should be appreciated that any suitable sensors are contemplated including, for example, a gyroscope, a gravity sensor, a rotation vector sensor, a magnetometer, a pressure sensor, and a location detection device (such as a GPS device or any device capable of measuring movement using cellular data or WiFi triangulation or any suitable alternative). In embodiments where the one or more sensors includes one or more accelerometers, the motion data samples can characterize a measurement of acceleration along one or more axes of movement. By measuring an amount of acceleration due to gravity, an accelerometer can determine its tilt angle relative to the earth. Additionally, by sensing an amount of dynamic acceleration, an accelerometer can determine how fast and in what direction the device is moving. In embodiments, a photoplethysmographic sensor may also be used to generate photoplethysmographic (PPG) data to calculate a heart rate, heart rate variability, and/or a respiration rate of the person P. A PPG sensor typically includes a light source, e.g., a light-emitting diode, and a photodetector and can be used to calculated a user's heart rate by measuring the time between peaks or by calculating a dominant frequency in the optical signal. A person's heart rate typically drops after the onset of sleep and continues to drop until early in the morning. The heart rate typically rises when the user wakes up or during short disturbances during sleep. Thus, these differences can be exploited using similar methods described herein.

As shown in FIG. 1A, the device 10 comprising one or more motion sensors can be a body-worn accelerometer configured to be worn at least partially around a person's wrist (e.g., a smartwatch). In alternate embodiments, the body-worn accelerometer can be configured to be worm at least partially around a person's forearm, upper arm, leg, or ankle, or any suitable part of the body. As shown in FIG. 1B, the device 10 comprising one or more motion sensors can be a patch having an adhesive component and an integrated sensor such that the patch can be secured directly to the person's skin. In alternate embodiments, the patch can be secured to the person's clothing.

A system 20 for classifying movement of a user during a period of low activity is also depicted in FIG. 1A. The system 20 includes a motion sensor analyzer 50, a motion level estimator 60, a low-temporal resolution representation adaptor 70, and a classifier 80. The motion sensor analyzer 50 is configured to receive raw sensor data from a motion sensor of device 10 and determine whether the data is stationary or not as further described herein. The motion level estimator 60 is configured to derive a low-temporal resolution representation of the sensor data based on a stationarity of the data. The low-temporal resolution representation adaptor 70 is configured to modify the low-temporal resolution representation to reduce sensitivity to potential sensor miscalibration and artifacts. System 20 further includes a classifier 80 configured to characterize the movement of the user during sleep based on the modified low-temporal resolution representation. Data representing the characterized movement can be outputted, for example, to a display, storage or a mobile device (shown schematically at 90 in FIG. 1A). The system 20 can be embodied within device 10 and can comprise one or more processors or microprocessors which execute appropriate software. The software could be downloaded and/or stored in a corresponding memory. Alternatively, the functional units of the system (e.g., the analyzers, estimators, adaptors, classifiers) may be implemented in the form of programmable logic. Generally, each functional unit of the system 20 can be implemented in the form of a circuit. The system 20 can also be implemented in a distributed manner involving different devices or apparatus. For example, the distribution may be in accordance with a client-server model.

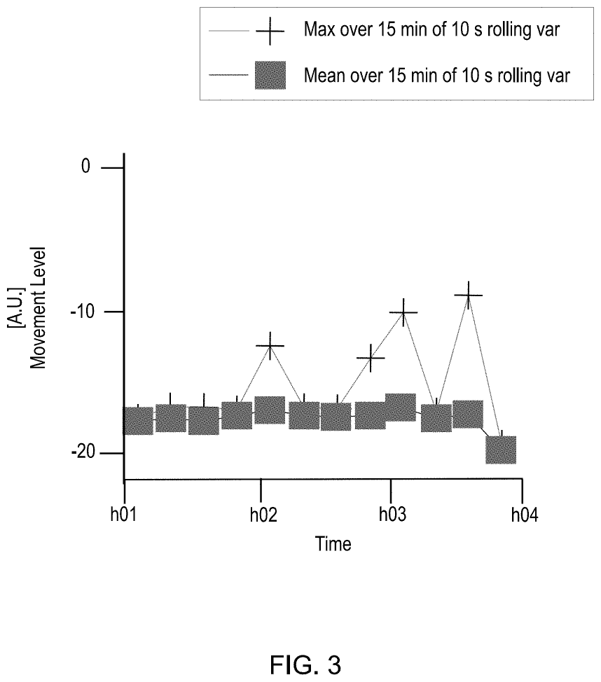

FIG. 3 shows example low-temporal resolution representations of the motion sensor data of FIG. 2. These low-temporal resolution representations are used to summarize the raw sensor data and save transmission bandwidth, storage capacity on device, and computational power as discussed above. For example, the raw sensor data of FIG. 2 can be analyzed by determining a movement level (e.g., a variance of accelerometer samples in a sliding window). In embodiments, a low-temporal resolution representation is based on determining a maximum over 15 minutes of 10 seconds rolling variance of the norm of the accelerometer signal. In embodiments, the low-temporal resolution representation is based on determining a mean over 15 minutes of 10 seconds rolling variance of the norm of the accelerometer signal. In still other embodiments, the low-temporal resolution representation is based on determining a median over 15 minutes of 10 seconds rolling variance of the norm of the accelerometer signal. In further embodiments, the low-temporal resolution representation is based on a count of movements per minute using accelerometer zero crossing measures. It should be appreciated that any suitable low-temporal resolution representations of the motion sensor data are contemplated. For example, other representations include 10 seconds rolling variance of the norm of the accelerometer signal, 10 seconds rolling median of the accelerometer signal, and the raw accelerometer signal.

Referring to FIG. 3, the low-time resolution representation includes (i) a maximum over 15 minutes of 10 seconds rolling variance of the norm of the accelerometer signal in FIG. 2, and (ii) a mean over 15 minutes of 10 seconds rolling variance of the norm of the accelerometer signal in FIG. 2. The features of the low-time resolution representation in FIG. 3 indicate several motion episodes around h02 and h03. The 15-minute means is slightly elevated, while the 15-minute maximum exhibits a large excursion. However, the full signal in FIG. 2 shows the absence of substantial motion during the periods of interest (e.g., episodes around h02 and h03). There is movement at h02 and h02, i.e., the person turns, but the signal before and after the turns can be seen to stay relatively constant. Thus, the low-time resolution representation of FIG. 3 does not accurately reflect motion in the accelerometer signal.

Body worn motion sensor accelerometers such as the motion sensor depicted in device 10 as used in the field can exhibit offset in their calibration and such calibration may drift over time. This miscalibration may induce “virtual” activity levels when the sensor changes orientation. This is caused by the change in measured size of the sensed gravity component with changing orientation. Similarly, movements may appear in different magnitude depending on movement direction and sensor orientation, such is aside from bias effects due to the offsets. These virtual activity levels may become of the same order of magnitude as the weak movements mentioned above. The weak motions (e.g., bed turns) together with miscalibration may lead to overestimated activity levels in a low temporal rate summary signal. Thus, the accuracy of a classifier can be affected in particular when the monitored movements are of a small size (e.g., low signal strengths). Although usually compensated, residual environmental temperature dependency for accelerometer bias is also expected.

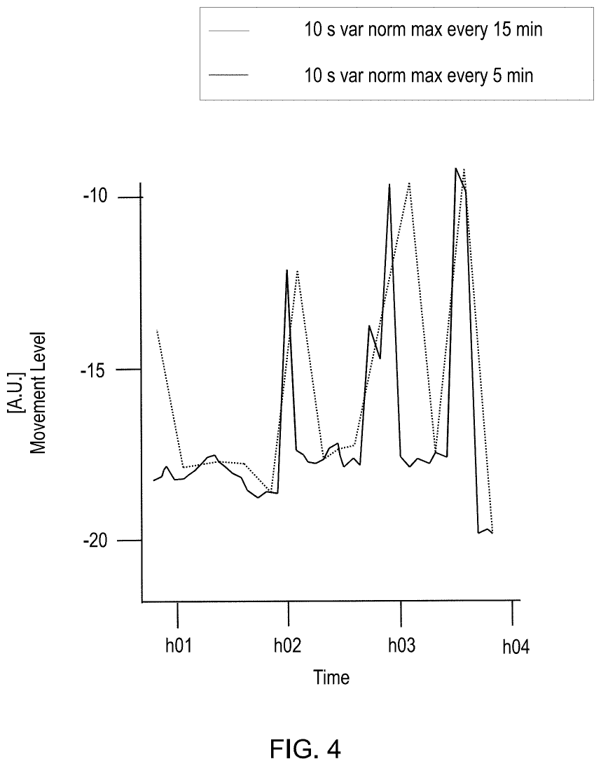

In not fully calibrated accelerometers, a rapid rotation may result in high (apparent) accelerometer norm variance due to the differences in offset in the two orientations. A rapid rotation refers to a rotation that occurs faster than the resolution of the low-temporal resolution representation. In case of long-term sensitivity drift or of incomplete temperature compensation, similar phenomena may arise. Addressing the problems directly with in-use calibration may not be possible under ordinary conditions. The high apparent motion could be minimized for instance by applying a non-linear filter to the raw sensor data before deriving the low temporal rate summary signal however, at a computational cost. FIG. 4 shows example rotation induced high (apparent) accelerometer norm variance in different low-temporal resolution settings. For example, the solid line in FIG. 4 represents apparent movement using a maximum over 5 minutes of 10 seconds rolling variance of the norm of the accelerometer signal. The dashed line in FIG. 4 represents apparent movement using a maximum over 15 minutes of 10 seconds rolling variance of the norm of the accelerometer signal. However, the full signal in FIG. 2 shows the absence of substantial apparent motion during the periods of interest. Thus, the low-temporal resolution representations of FIG. 4 are not representative of motion in the accelerometer signal.

The magnitude of spurious motion level in a low temporal rate summary signal depends on a number of facts, mostly on the exact time of the orientation change during the time interval as maximum and mean are not robust statistical indicators. Although the use of robust statistical indicators might render the effect of the spurious motion on the low temporal rate summary signal more systematic and thus, easier to compensate, some residual information loss is intrinsic to the rate lowering operation. The potentially lost information might be useful for a number of use cases (e.g., estimating quality parameters during bed turns).

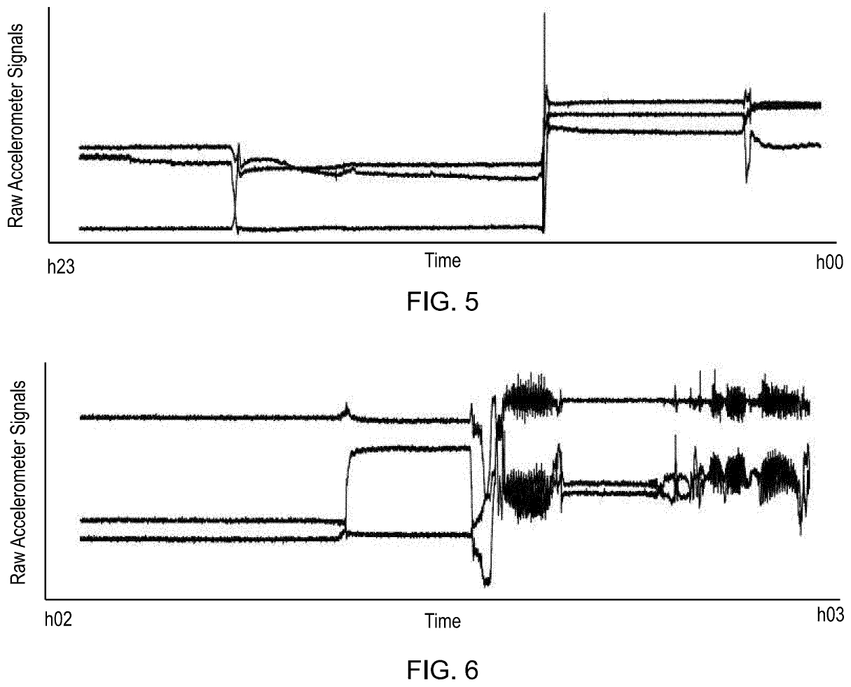

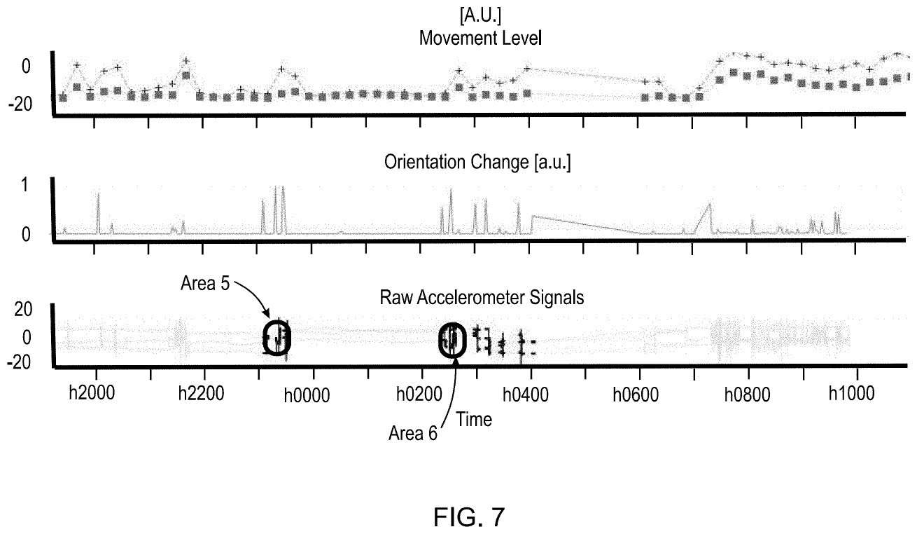

FIG. 5 shows an example full tri-axial accelerometer signal recorded during one bed turn. FIG. 6 shows an example full tri-axial accelerometer signal recorded during sporadic nocturnal activity. Despite the stark differences in the full tri-axial accelerometer signals in FIGS. 5 and 6, both yield substantially similar features in the low-temporal resolution representation shown in the top panel of FIG. 7. As shown in the bottom panel of FIG. 7 depicting raw accelerometer signals, area 5 corresponds to the accelerometer signal of one bed turn shown in FIG. 5. Area 6 in the bottom panel of FIG. 7 corresponds to the accelerometer signal of sporadic nocturnal activity shown in FIG. 6. Again, despite the two very different scenarios represented in FIGS. 5 and 6, both generate similar motion levels in the low-temporal resolution representation in the top panel of FIG. 7. Thus, user behavior can make it challenging to accurately classify small bodily movements like the miscalibration discussed above.

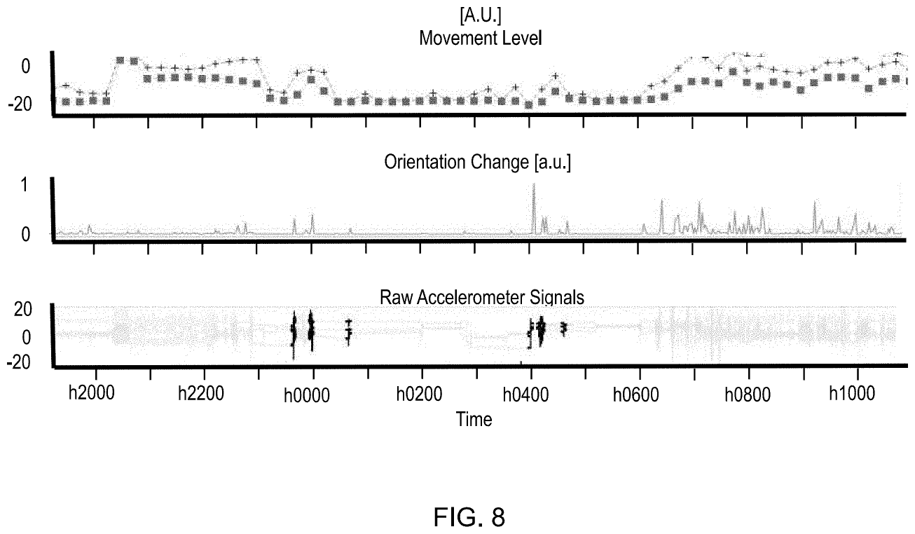

FIGS. 7 and 8 show low-temporal resolution features, orientation data, and raw accelerometer signals for bodily movement during sleep for different users. In FIG. 7, a data gap is present between h0400 and h0600 due to the motion sensor device being turned off. In the top panel of FIG. 7, the low time resolution representation includes maximum values over 15-minute periods of 10 seconds rolling variance of the norm and mean values over 15-minute periods of 10 seconds rolling variance of the norm. The maximum and mean values are compared with 1 minute temporally spaced raw accelerometer samples to identify rotation periods of the raw accelerometer data. Based on the comparison, portions of the raw accelerometer data, such as areas 5 and 6, and the other emphasized portions in the bottom panel can be selected and separately preprocessed and/or differently stored. In FIG. 8 there is no data gap as there is in FIG. 7. The top panel of FIG. 8 shows a low time resolution representation of maximum and mean values over 15-minute periods of 10 seconds rolling variance of the norm. These values are also compared with 1 minute temporally spaced raw accelerometer samples to identify rotation periods of the raw accelerometer data. Based on the comparison, portions of the raw accelerometer data can be selected and separately preprocessed and/or differently stored. The particular portions of the raw accelerometer data that are selected are highlighted in FIGS. 7 and 8. While the movements visible in the low time resolution representation of FIG. 7 (top panel) can be deemed sporadic bed turns, some of the movements visible in the low time resolution representation of FIG. 8 (top panel) can be deemed to be actual subject movements that may be clinically relevant. As described further below, the sporadic and continuous movement episodes (e.g., bed movement vs. short awakening and walking) can be differentiated by using multiple resolution approaches.

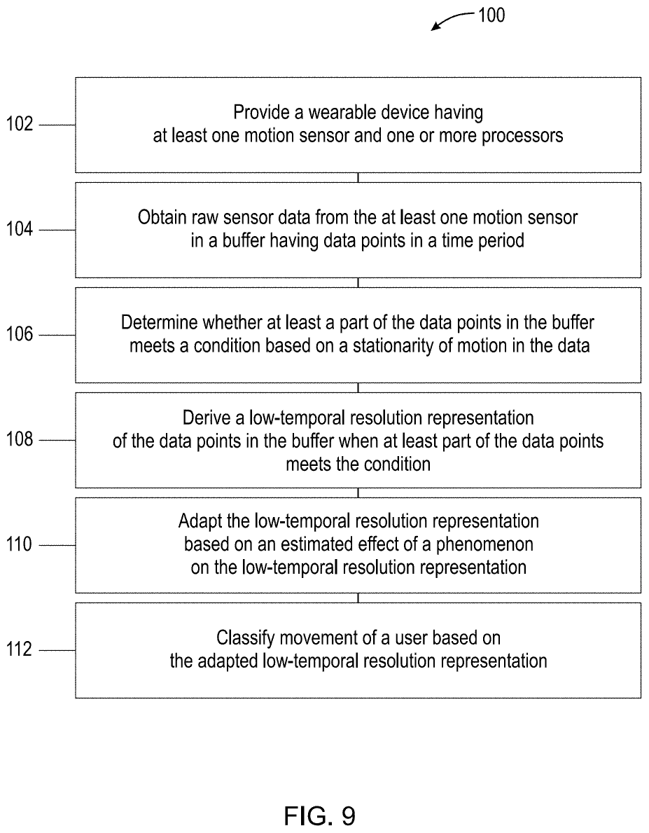

Referring to FIG. 9, an example process 100 of classifying movement of a user during a period of low activity is provided. At step 102, a wearable device such as device 10 as described herein is provided. The wearable device is configured to be worn by the user and includes one or more motion sensors and one or more processors. In embodiments, the wearable device includes a single body worn sensor, a plurality of body worn sensors of the same type, or two or more body worn sensors of at least two different types. Each of the one or more sensors can benefit from the adaptive low-temporal resolution representation scheme individually. The one or more sensors can also benefit from using a sensor fusion scheme to combine low-temporal resolution representations of different sensors in a similar adaptive way.

At step 104, the one or more processors of the wearable device obtains or receives raw sensor data from the one or more motion sensors of the wearable device. In embodiments, the motion sensor(s) automatically transmit the raw sensor data continuously or at predefined intervals to the one or more processors. In embodiments, the motion sensor(s) transmit the raw sensor data when requested. The obtained or received raw sensor data can be embodied in a buffer having a plurality of sensor data points within a time period. For example, the buffer can include a sequence of raw data points (x₁, . . . , xₑ) taken at equally spaced points in time from time t=1 to time t=e.

At step 106, the one or more processors determine (e.g., via motion sensor analyzer 50) whether data points in a buffer of the raw sensor data meet a predefined condition based on a stationarity of the motion in the raw sensor data. All of the data points in the buffer can be analyzed together or the data points can be analyzed in subsets. The term “stationarity” refers to statistical stationarity and whether the statistical properties of the motion in the period (e.g., mean, variance, autocorrelation, spectral shape etc.) are constant over the period or not. The term “stationarity” is generally understood as the signal exhibiting constant mean and constant autocorrelation (autocorrelation depends on time gap). In other embodiments, other aspects might be tested to decide whether the signal is stationary or not. For example, only the variance (=autocorrelation at zero time gap) is tested, or spectral shape (=Fourier Transform of autocorrelation) is tested. In embodiments, the predefined condition indicates that the period of motion in the raw sensor data was stationary. In other embodiments, the predefined condition indicates that the period of motion in the raw sensor data was not stationary. In embodiments, the data in the buffer is evaluated using historical low time resolution representation data available from the device 10. For example, the offset per orientation can be estimated based on recordings of the accelerometer single channel values in the epochs with the lowest accelerometer normal motion levels. In another example, expected motion artifacts due to offset changes can be estimated. In embodiments, the expected motion artifact amplitude can be calculated as 0.5*Max_expected_offset. In common operating conditions for mobile help button devices, for example, 10*log 10(0.5*0.2(m/s²)²)˜−7 dB, 10*np.log 10(0.5*0.4)˜−4 dB (at +−8 g dynamic range @ 10 bit ACDC>LSB˜0.01 ms).

In embodiments where the predefined condition indicates that the period of motion in the raw sensor data was not stationary, step 106 is used to flag the buffer or one or more subsets of the buffer as different from other motion periods. The flagged buffer or buffer subset(s) can subsequently be processed with one or more algorithms to account for this difference in sleep detection steps.

In embodiments where the predefined condition indicates that the period of motion in the raw sensor data was stationary, the one or more processors at step 108 derive a low-temporal resolution representation of the raw sensor data points in the buffer when at least part of the raw sensor data points meets the predefined condition based on stationarity. The step of deriving the low-temporal resolution representation can include estimating a motion level as depicted graphically in FIGS. 3 and 4 e.g., via motion level estimator 60. For example, the low-temporal resolution representation can be based on maximum values over 15 minutes of 10 seconds rolling variance of the norm of the accelerometer signal or any of the alternatives discussed above. In embodiments, the low-temporal resolution representation can be stored in a memory of the device 10.

In step 110, the one or more processors adapts the low-temporal resolution representation (e.g., via the low-temporal resolution representation adaptor 70). In example embodiments, historical low-temporal resolution representation data can be used to evaluate the effect of orientation changes on accelerometer offset. The one or more processors can retrieve the historical data from a memory of the device 10. Alternatively, the one or more processors can retrieve the historical data from a memory of a separate device remote from the body-worn device 10. Using the retrieved historical data, the one or more processors can estimate an effect of a phenomenon (e.g., offset) on the low-temporal resolution representation and adapt the low-temporal resolution representation derived in step 108 accordingly.

In embodiments, if the predefined condition determined in step 106 indicates that the period of motion in the raw sensor data was stationary, the one or more processors can generate an alternative representation of the raw sensor data. The alternative representation can be more detailed and representative of the original signal. In embodiments, the alternative representation is the raw motion signal. In other embodiments, the alternative representation can be another different low-temporal resolution representation having a resolution that is larger than the resolution of the low-temporal resolution representation derived in step 108 yet smaller than the resolution of the raw sensor data (e.g., 50 Hz). In embodiments, the alternative representation is a low-temporal resolution representation from one or more individual axes of a tri-axial sensor. The alternative representation can be a low-temporal resolution representation of a signal from another sensor which can be of the same type or of a different type in embodiments. In still other embodiments, the alternative representation can be another different low-temporal resolution representation of the same time resolution as derived in step 108 but including different indicators (e.g., spread in variance of 10 second windows within a 15-minute buffer or a variance of each accelerometer channel). In embodiments, the same alternative representation from consecutive groups of samples are concatenated in order to achieve a sampling rate that is constant over a longer time scale. In embodiments, the step of generating an alternative representation of the raw sensor data can be repeated for subsequent buffers without checking the stationarity of each individual period.

In embodiments, one or more conditions can be evaluated to choose which alternative representation should be used. One example condition can be a change in most significant bits of accelerometer samples indicative of large orientation change in embodiments. In other embodiments, the one or more conditions can include dot product values between first and last samples of the buffer or a buffer subset indicative of orientation change. In still other embodiments, the one or more conditions can be based on the high values of low-frequency bins in a Fast-Fourier Transform (FFT) of the accelerometer signal. Such high values are indicative of rotation in the accelerometer signal. In further embodiments, the one or more conditions can be a uniformity of the motion level within the samples of interest in the accelerometer signal.

The one or more conditions can also include a determination of whether a variance in values of summary statistics meets or exceeds some predetermined threshold. For example, the one or more processors can calculate and analyze values of summary statistics of the raw sensor data and determine that the values exhibit a variance that is below a predetermined threshold (e.g., 0.01 (m/s²)²) depending on accelerometer noise floor and dynamic range. The one or more processors can also determine whether the values exhibit a variance that meet or exceeds the same predetermined threshold. In embodiments, two or more predetermined thresholds can be used. For example, a variance of the values of the summary statistics can be deemed low if the variance is below a first predetermined threshold (e.g., 0.01 (m/s²)²). If the variance is above the first predetermined threshold but below a second predetermined threshold (e.g., 0.03 (m/s²)²), then the variance can be deemed intermediate. The one or more processors can also determine that the variance meets or exceeds the second predetermined threshold. Different alternative representations can be used depending on the variance.

One particular embodiment of a method for classifying movement of a user during a period of low activity is as follows. A body-worn device including motion sensors records raw sensor data of a period of low activity. At least part of the raw sensor data in the period of low activity is accumulated in a first buffer from which summary statistics are calculated (e.g., variance of the acceleration norm of the last 10 recorded seconds). The low-temporal resolution representation described herein is one embodiment of the summary statistics calculated or derived for the first buffer. Such a representation of the first buffer is then accumulated in a second buffer of additional raw sensor data. In embodiments, the second buffer can be related to a longer time scale (e.g., 15 minutes which is 900 times the previous first buffer). When the second buffer is complete, the variance of the values of the summary statistics is computed for the second buffer. In the case where the second buffer is 900 times the previous buffer, a max and mean of the 900 buffers can be computed. If the variance of the second buffer is below a predetermined threshold (e.g., 0.01 (m/s²)²), it is determined that the period corresponding to the second buffer consists of uniform activity and the summary statistics embodied as the low-temporal resolution representation provides an accurate representation of the user's activity. Thus, in this embodiment the low-temporal resolution is stored and the process repeats for subsequent buffers to see if the uniform activity continues. If on the other hand, the variance of the second buffer meets or exceeds the predetermined threshold, it is determined that the period corresponding to the second buffer includes an indication of sporadic motion and all the values from the first buffer are stored rather than the summary statistics. The values of the first buffer are used to provide additional detail on the movement. In embodiments, the values of the first buffer can further be stored for additional periods of activity as well.

In an embodiment including two predetermined thresholds, if the variance of the second buffer is below a first predetermined threshold (e.g., 0.01 (m/s²)²), it is determined that the period corresponding to the second buffer consists of uniform activity and the summary statistics embodied as the low-temporal resolution representation of the first buffer can be used as it provides an accurate representation of the user's activity. In this scenario, the low-temporal resolution representation of the first buffer is stored. If the variance of the second buffer is above the first predetermined threshold (e.g., 0.01 (m/s²)²) but below a second predetermined threshold (e.g., 0.03 (m/s²)²), it is determined that the period corresponding to the second buffer provides an indication of a possible bed turn and a low-temporal resolution representation of the individual accelerometer axes are stored instead. If the variance of the second buffer is above the second predetermined threshold, it is determined that the period corresponding to the second buffer provides an indication of sporadic motion and all of the values from the first buffer are stored instead.

Once the low-temporal resolution representation is adapted and/or an alternate representation is chosen, at step 110 the one or more processors proceeds to classify the movement based on the adapted or alternate representation.

Thus, the present disclosure allows for the use of summarized motion data when there is uniform activity and motion data when there is an indication of some sporadic motion. The storage space of a body-worn sleep tracking device is enhanced because of the multi-scale signal storage; detailed signals are used only when needed. Embodiments described herein implement this multi-resolution signal storage. Alternate embodiments described herein use variance or some suitable equivalent to flag one or more parts of the data that can be subjected to further processing subsequently.

As described above, deriving and adapting the low time resolution representation of raw sensor data removes the virtual activity levels from the low-rate summary signal, such that weak movements remain. Advantageously, removing these virtual activity levels reduces the sporadic night movements reported to the user or a caregiver due to bed turns. In this way, the activity-level resolution is raised, leading to improved accuracy of a subsequent activity classifier.

Using the methods and systems described herein, an improved time resolution is achieved, as well as an improved amplitude resolution (sensitivity) may be achieved (when non-linear metrics such as variance are used), while reducing the sensitivity to potential sensor miscalibration and reducing artifacts.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for classifying movement of a user during a period of low activity, the method comprising the steps of:
providing a wearable device configured to be worn by the user, the wearable device having at least one motion sensor and one or more processors;
obtaining, via the one or more processors, raw sensor data from the at least one motion sensor in a first buffer, the first buffer comprising a first plurality of raw sensor data points within a first time period;
determining, via the one or more processors, whether at least part of the plurality of raw sensor data points in the first buffer meets a predefined condition based on a stationarity of motion in the raw sensor data;
deriving, via the one or more processors, a low-temporal resolution representation of the raw sensor data points in the first buffer when the at least part of the plurality of raw sensor data points in the first buffer meets the predefined condition based on stationarity;
adapting, via the one or more processors, the low-temporal resolution representation based on an estimated effect of a phenomenon on the low-temporal resolution representation; and
classifying movement of the user based on the adapted low-temporal resolution representation.

2. The method of claim 1, further comprising the steps of:
retrieving historical low-temporal resolution representation data; and
estimating the effect of the phenomenon on the low-temporal resolution representation based on the historical low-temporal resolution representation data.

3. The method of claim 1, wherein the step of determining whether the plurality of raw sensor data points meet the predefined condition comprises evaluating the predefined condition using historical low-temporal resolution representation data from the wearable device.

4. The method of claim 1, further comprising the steps of:
storing the low-temporal resolution representation on the one or more processors of the wearable device; and
transmitting the low-temporal resolution representation for processing to classify the movement of the user.

5. The method of claim 1, further comprising the step of deriving, via the one or more processors, an alternative representation of the raw sensor data points after the step of deriving the low-temporal resolution representation.

6. The method of claim 5, further comprising the step of storing the alternative representation on the one or more processors of the wearable device.

7. The method of claim 1, further comprising the step of flagging at least part of the first buffer as different when the predefined condition indicates the buffer comprises non-stationary movement.

8. The method of claim 1, further comprising the steps of:
obtaining, via the one or more processors, raw sensor data from the at least one motion sensor in a second buffer, the second buffer comprising a second plurality of raw sensor data points within a second time period; and
storing the low-temporal resolution representation in the second buffer.

9. The method of claim 8, further comprising the steps of:
determining a variance of the second plurality of raw sensor data points based on the low-temporal resolution representation stored in the second buffer; and
storing the derived and adapted low-temporal resolution representation when the determined variance is below a predetermined threshold value.

10. The method of claim 8, further comprising the steps of:
determining a variance of the second plurality of raw sensor data points based on the low-temporal resolution representation stored in the second buffer; and
storing the first plurality of raw sensor data points from the first buffer when the determined variance is above a predetermined threshold value.

11. A system for classifying movement of a user (P) during a period of low activity, comprising:
a wearable device configured to be worn by the user, the wearable device comprising at least one motion sensor; and
one or more processors communicably coupled with the at least one motion sensor and configured to:
obtain raw sensor data from the at least one motion sensor in a first buffer, the first buffer comprising a first plurality of raw sensor data points within a first time period;
determine whether at least part of the plurality of raw sensor data points in the first buffer meets a predefined condition based on a stationarity of motion in the raw sensor data;
derive a low-temporal resolution representation of the raw sensor data points in the first buffer when the at least part of the plurality of raw sensor data points in the first buffer meets the predefined condition based on stationarity;
adapt the low-temporal resolution representation based on an estimated effect of a phenomenon on the low-temporal resolution representation; and
classify movement of the user based on the adapted low-temporal resolution representation.

12. The system of claim 11, wherein the one or more processors are configured to:
store the low-temporal resolution representation; and
transmit the low-temporal resolution representation for processing to classify the movement of the user.

13. The system of claim 11, wherein the one or more processors are configured to:
derive an alternative representation of the raw sensor data points based on the low-temporal resolution representation; and
store the alternative representation.

14. The system of claim 11, wherein the one or more processors are configured to:
obtain raw sensor data from the at least one motion sensor in a second buffer, the second buffer comprising a second plurality of raw sensor data points within a second time period; and
store the low-temporal resolution representation in the second buffer.

15. The system of claim 14, wherein the one or more processors are configured to:
determine a variance of the second plurality of raw sensor data points based on the low-temporal resolution representation stored in the second buffer; and
store the derived and adapted low-temporal resolution representation when the determined variance is below a predetermined threshold value, or
store the first plurality of raw sensor data points from the first buffer when the determined variance is above the predetermined threshold value.
